(19) United States
(12) Patent Application Publication
(10) Pub. No.: US 2025/0282793 A1
(43) Pub. Date: Sep. 11, 2025

(54) COMPOUND

(71) Applicant: Sumitomo Chemical Co., Ltd., Tokyo (JP)

(72) Inventors: Connor William Patrick, Godmanchester (GB); Michal Robert Maciejczyk, Godmanchester (GB)

(73) Assignee: Sumitomo Chemical Co., Ltd., Tokyo (JP)

(21) Appl. No.: 18/745,867

(22) Filed: Jun. 17, 2024

(30) Foreign Application Priority Data
Mar. 8, 2024 (GB) 2403414.2

(51) Int. Cl. C07D 495/22; H10K 50/814; H10K 50/82; H10K 85/60

(52) U.S. Cl. CPC C07D 495/22; H10K 50/814; H10K 50/82; H10K 85/615; H10K 85/6576

(57) ABSTRACT

A compound of formula (I):

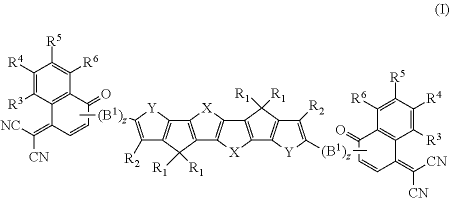

R¹ in each occurrence is independently a substituent; R² in each occurrence is H or substituent; R³-R⁶ are each independently in each occurrence H or a halogen; X in each occurrence is independently selected from is O, S and NR³ wherein R³ is H or a substituent; Y in each occurrence is independently O or S; B¹ independently in each occurrence is a bridging group; and z in each occurrence is independently 0, 1, 2 or 3. The compound of formula (I) may be used as an electron-accepting material of an organic photoresponsive device.

### BACKGROUND

Embodiments of the present disclosure relate to electron-accepting compounds and more specifically compounds suitable for use as an electron-accepting material in a photoresponsive device.

An organic photoresponsive device may contain a photoactive layer of an electron-donating material and an electron-accepting material between an anode and a cathode. Known electron-accepting materials include fullerenes and non-fullerene acceptors (NFAs).

Tengfei Li et al, “Sensitive photodetection below silicon bandgap using quinoid-capped organic semiconductors” Sci. Adv. 9, eadf6152 (2023) discloses quinoid-capped compounds for use in near infrared organic photodetectors.

CN116425768 discloses near-infrared absorbing conjugated macromolecules with quinoid terminal groups of formula (I):

### SUMMARY

The present disclosure provides a compound of formula (I):

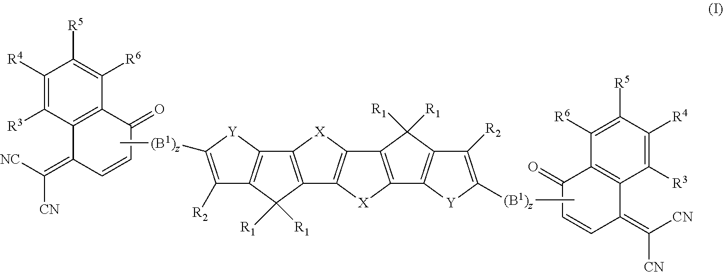

wherein:
R¹ in each occurrence is independently a substituent;
R² in each occurrence is H or substituent;
R³-R⁶ are each independently in each occurrence H or a halogen;
X in each occurrence is independently selected from is O, S and NR³ wherein R³ is H or a substituent;
Y in each occurrence is independently O or S;
B¹ independently in each occurrence is a bridging group; and
z in each occurrence is independently 0, 1, 2 or 3.

Preferably, each R¹ is independently selected from the group consisting of:
linear, branched or cyclic C₁₋₂₀ alkyl wherein one or more non-adjacent C atoms may be replaced by O, S, NR⁷, CO or COO wherein R⁷ is H or a C₁₋₁₂ hydrocarbyl and one or more H atoms of the C₁₋₂₀ alkyl may be replaced with F; and
a group of formula (Ak)u-(Ar⁷)v wherein Ak is a C₁₋₂₀ alkylene chain in which one or more non-adjacent C atoms may be replaced with O, S, NR⁷, CO or COO; u is 0 or 1; Ar⁷ in each occurrence is independently an aromatic or heteroaromatic group which is unsubstituted or substituted with one or more substituents; and v is at least 1, optionally 1, 2 or 3.

Substituents of Ar⁷, if present, are preferably selected from F; Cl; NO₂; CN; and C₁₋₂₀ alkyl wherein one or more non-adjacent C atoms may be replaced with O, S, NR⁷, CO or COO and one or more H atoms may be replaced with F.

Optionally, Ar⁷ is a C₆₋₂₀ aryl, optionally phenyl.

Optionally, each R² is independently selected from H; F; Cl; NO₂; CN; and C₁₋₂₀ alkyl wherein one or more non-adjacent C atoms may be replaced with O, S, NR⁷, CO or COO and one or more H atoms may be replaced with F. Optionally, each R² is H.

In some embodiments, u is 0 and v is 1, i.e., R¹ is a group of formula Ar⁷.

Optionally, R³ and R⁶ are each H.

Optionally, at least one of R⁴ and R⁵ is a halogen.

Optionally, R³-R⁶ are each independently in each occurrence selected from H, F and Cl.

Optionally, at least one z is at least 1.

Optionally, B¹ in each occurrence is selected from vinylene; thiophene; furan; thienothiophene; furofuran; and thienofuran.

Optionally, the compound of formula (I) is a compound of formula (Ia):

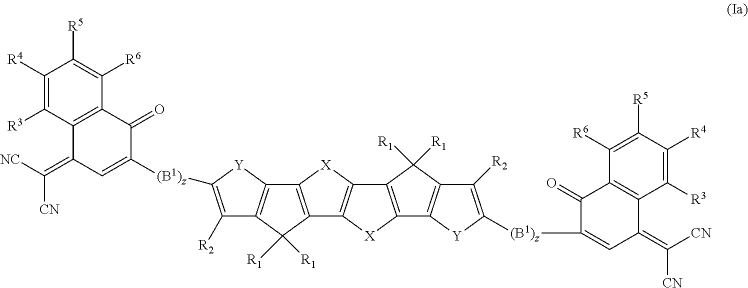

The present disclosure provides a composition comprising an electron-donating material and an electron-accepting material wherein the electron accepting material is a compound of formula (I).

The present disclosure provides an organic electronic device comprising an active layer comprising a compound of formula (I).

Optionally, the organic electronic device comprises an active layer comprising a composition as described herein.

Optionally, the organic electronic device is an organic photoresponsive device and wherein the active layer is a photoactive disposed between the anode and cathode. Optionally, the photoactive layer is a bulk heterojunction layer comprising a composition as described herein.

Optionally, the organic photoresponsive device is an organic photodetector.

The present disclosure provides a photosensor comprising a light source and an organic photodetector as described herein, wherein the photosensor is configured to detect light emitted from the light source. Optionally, the light source emits light having a peak wavelength of greater than 900 nm.

The present disclosure provides a formulation comprising a compound formula (I) or a composition comprising an electron-accepting compound of formula (I) and an electron-donating compound dissolved or dispersed in one or more solvents.

The present disclosure provides a method of forming an organic electronic device as described herein, wherein formation of the active layer comprises deposition of a formulation as described herein onto a surface and evaporation of the one or more solvents.

### DESCRIPTION OF THE DRAWINGS

FIG. 1 is a schematic illustration of an organic photoresponsive device according to some embodiments of the present disclosure;

FIG. 2 shows film and solution absorption spectra of Compound Example 1;

FIG. 3 shows external quantum efficiencies vs wavelength of organic photodetectors containing Compound Example 1 and different donor polymers;

FIG. 4 shows current densities vs voltage for the OPDs of FIG. 3;

FIG. 5 shows specific detectivity (D*) vs wavelength for the OPDs of FIGS. 3 and 4; and

FIG. 6 shows specific detectivity contours at 1300 nm for the OPDs of FIGS. 3 and 4.

The drawings are not drawn to scale and have various viewpoints and perspectives. The drawings are some implementations and examples. Additionally, some components and/or operations may be separated into different blocks or combined into a single block for the purposes of discussion of some of the embodiments of the disclosed technology. Moreover, while the technology is amenable to various modifications and alternative forms, specific embodiments have been shown by way of example in the drawings and are described in detail below. The intention, however, is not to limit the technology to the particular implementations described. On the contrary, the technology is intended to cover all modifications, equivalents, and alternatives falling within the scope of the technology as defined by the appended claims.

### DETAILED DESCRIPTION

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” Additionally, the words “herein,” “above,” “below,” and words of similar import, when used in this application, refer to this application as a whole and not to any particular portions of this application. Where the context permits, words in the Detailed Description using the singular or plural number may also include the plural or singular number respectively. The word “or,” in reference to a list of two or more items, covers all of the following interpretations of the word: any of the items in the list, all of the items in the list, and any combination of the items in the list. References to a layer “over” another layer when used in this application means that the layers may be in direct contact or one or more intervening layers may be present. References to a layer “on” another layer when used in this application means that the layers are in direct contact. References to a specific element of the Periodic Table include any isotope of that element unless specifically stated otherwise.

The teachings of the technology provided herein can be applied to other systems, not necessarily the system described below. The elements and acts of the various examples described below can be combined to provide further implementations of the technology. Some alternative implementations of the technology may include not only additional elements to those implementations noted below, but also may include fewer elements.

These and other changes can be made to the technology in light of the following detailed description. While the description describes certain examples of the technology, and describes the best mode contemplated, no matter how detailed the description appears, the technology can be practiced in many ways. As noted above, particular terminology used when describing certain features or aspects of the technology should not be taken to imply that the terminology is being redefined herein to be restricted to any specific characteristics, features, or aspects of the technology with which that terminology is associated. In general, the terms used in the following claims should not be construed to limit the technology to the specific examples disclosed in the specification, unless the Detailed Description section explicitly defines such terms. Accordingly, the actual scope of the technology encompasses not only the disclosed examples, but also all equivalent ways of practicing or implementing the technology under the claims.

To reduce the number of claims, certain aspects of the technology are presented below in certain claim forms, but the applicant contemplates the various aspects of the technology in any number of claim forms.

In the following description, for the purposes of explanation, numerous specific details are set forth in order to provide a thorough understanding of implementations of the disclosed technology. It will be apparent, however, to one skilled in the art that embodiments of the disclosed technology may be practiced without some of these specific details.

### Bridging Units

In the case where z is 1, 2 or 3, bridging units B¹ are preferably each selected from vinylene, arylene, heteroarylene, arylenevinylene and heteroarylenevinylene wherein the arylene and heteroarylene groups are monocyclic or bicyclic groups, each of which may be unsubstituted or substituted with one or more substituents.

Optionally, B¹ is selected from units of formulae (VIa)-(VIo):

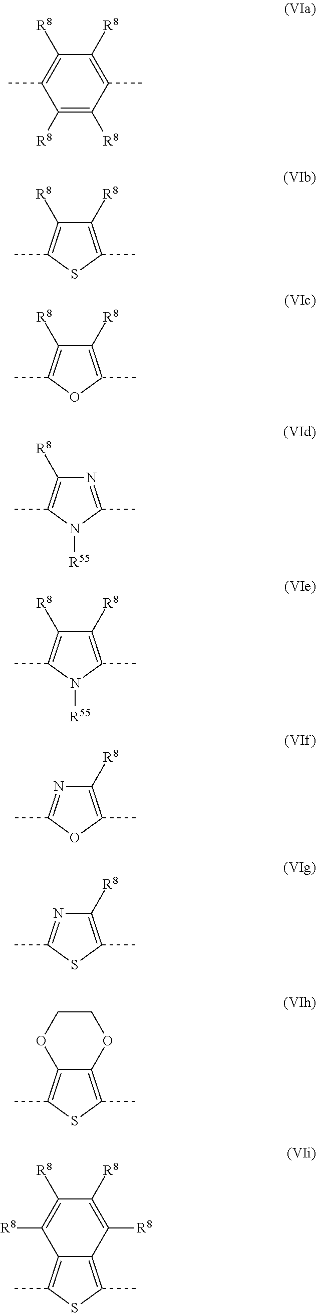

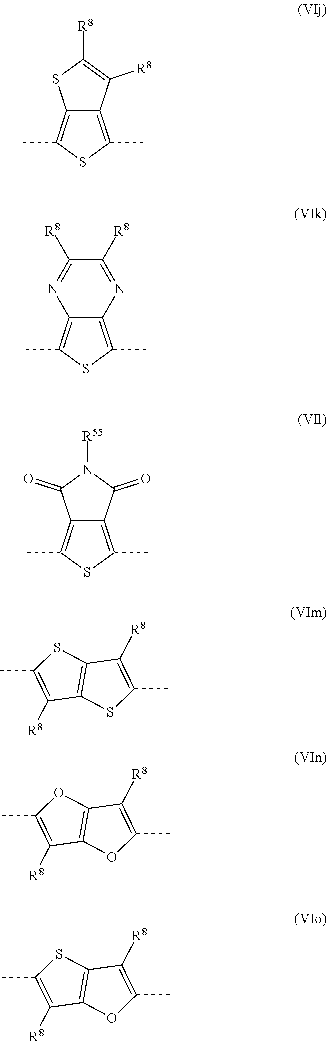

wherein R⁵⁵ is H or a substituent, optionally H or a C₁₋₂₀ hydrocarbyl group; and R⁸ in each occurrence is independently H or a substituent, preferably H or a substituent selected from F; CN; NO₂; C₁₋₂₀ alkyl wherein one or more non-adjacent C atoms may be replaced with O, S, NR⁷, COO or CO and one or more H atoms of the alkyl may be replaced with F, wherein R⁷ is H or a C₁₋₂₀ hydrocarbyl group; and phenyl which is unsubstituted or substituted with one or more substituents.

Exemplary substituents of a phenyl group R⁵⁵ are F; CN; NO₂; C₁₋₂₀ alkyl wherein one or more non-adjacent C atoms may be replaced with O, S, NR⁷, COO or CO and one or more H atoms of the alkyl may be replaced with F

A C₁₋₂₀ hydrocarbyl group as described anywhere herein may be selected from C₁₋₂₀ alkyl; and phenyl which is optionally substituted with one or more C₁₋₁₂ alkyl groups.

R⁸ groups of formulae (VIa), (VIb) and (VIc) may be linked to form a bicyclic ring which may be substituted with one or more substituents, optionally one or more substituents selected from F; CN; NO₂; C₁₋₂₀ alkyl wherein one or more non-adjacent C atoms may be replaced with O, S, NR⁷, COO or CO and one or more H atoms of the alkyl may be replaced with F.

R⁸ is preferably H, C₁₋₂₀ alkyl or C₁₋₁₉ alkoxy.

R⁸ groups of formulae (VIa), (VIb) and (VIc) may be linked to form an optionally substituted bicyclic ring.

### Electron-Donating Material

The present disclosure provides a composition comprising an electron-accepting compound of formula (I) and an electron-donating material. The composition may consist of the compound of formula (I) and the electron-donating material. The composition may comprise one or more further materials, for example one or more further electron-accepting compounds or one or more electron-donating compounds.

In some embodiments, the weight of the electron-donating material(s) to the electron-accepting material(s) is from about 1:0.5 to about 1:2. Optionally, the weight of the electron-accepting material or materials is less than the weight of the electron-donating material(s).

Preferably, the electron-donating material has a type II interface with the electron-accepting material of formula (I), i.e., the electron-donating material has a shallower HOMO and LUMO that the corresponding HOMO and LUMO levels of both the fullerene and the NFA of the electron-accepting compound of formula (I). Preferably, the compound of formula (I) has a HOMO level that is at least 0.05 eV deeper, optionally at least 0.10 eV deeper, than the HOMO of the electron-donating material.

Optionally, the gap between the HOMO level of the electron-donating material and the LUMO level of the electron-accepting compound of formula (I) is less than 1.4 eV.

Exemplary electron-donating materials of a photoactive layer as described herein are disclosed in, for example, WO2013/051676, the contents of which are incorporated herein by reference.

The electron-donating material may be a non-polymeric or polymeric material.

In a preferred embodiment the electron-donating material is an organic conjugated polymer, which can be a homopolymer or copolymer including alternating, random or block copolymers. The conjugated polymer is preferably a donor-acceptor polymer comprising alternating electron-donating repeat units and electron-accepting repeat units.

Preferred are non-crystalline or semi-crystalline conjugated organic polymers.

Further preferably the electron-donating polymer is a conjugated organic polymer with a low bandgap, typically between 2.5 eV and 1.5 eV, preferably between 2.3 eV and 1.8 eV.

Optionally, the electron-donating polymer has a HOMO level no more than 5.5 eV from vacuum level. Optionally, the electron-donating polymer has a HOMO level at least 4.1 eV from vacuum level. As exemplary electron-donating polymers, polymers selected from conjugated hydrocarbon or heterocyclic polymers including polyacene, polyaniline, polyazulene, polybenzofuran, polyfluorene, polyfuran, polyindenofluorene, polyindole, polyphenylene, polypyrazoline, polypyrene, polypyridazine, polypyridine, polytriarylamine, poly(phenylene vinylene), poly(3-substituted thiophene), poly(3,4-bisubstituted thiophene), polyselenophene, poly(3-substituted selenophene), poly(3,4-bisubstituted selenophene), poly(bisthiophene), poly(terthiophene), poly(bisselenophene), poly(terselenophene), polythieno[2,3-b]thiophene, polythieno[3,2-b]thiophene, polybenzothiophene, polybenzo[1,2-b: 4,5-b′]dithiophene, polyisothianaphthene, poly(monosubstituted pyrrole), poly(3,4-bisubstituted pyrrole), poly-1,3,4-oxadiazoles, polyisothianaphthene, derivatives and co-polymers thereof may be mentioned.

Preferred examples of donor polymers are copolymers of polyfluorenes and polythiophenes, each of which may be substituted, and polymers comprising benzothiadiazole-based and thiophene-based repeating units, each of which may be substituted.

The donor polymer is preferably a donor-acceptor (DA) copolymer comprising a donor repeat unit and an acceptor repeat unit.

Preferred donor units are selected from thiophene which is optionally substituted with one or more substituents R¹¹ as described above; and repeat units of formulae (X), (XII) and (XII):

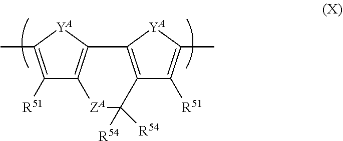

wherein:
Y^{A} in each occurrence is independently O, S or NR⁵⁵; Z^{A} in each occurrence is O, CO, S, NR⁵⁵ or C(R⁵⁴) 2; and R⁵¹, R⁵⁴ and R⁵⁵ independently in each occurrence is H or a substituent.

Optionally, R⁵¹ independently in each occurrence is selected from H; F; C₁₋₂₀ alkyl wherein one or more non-adjacent C atoms may be replaced with O, S, NR⁷, COO or CO and one or more H atoms of the alkyl may be replaced with F; and an aromatic or heteroaromatic group Ar³ which is unsubstituted or substituted with one or more substituents.

In some embodiments, Ar³ may be an aromatic group, e.g., phenyl. The one or more substituents of Ar³, if present, may be selected from C₁₋₁₂ alkyl wherein one or more non-adjacent C atoms may be replaced with O, S, NR⁷, COO or CO and one or more H atoms of the alkyl may be replaced with F.

Preferably, each R⁵¹ is H.

Preferably, each R⁵⁴ is selected from the group consisting of:
H;
F;
linear, branched or cyclic C₁₋₂₀ alkyl wherein one or more non-adjacent C atoms may be replaced by O, S, NR⁷, CO or COO and one or more H atoms of the C₁₋₂₀ alkyl may be replaced with F; and
a group of formula (Ak)u-(Ar⁷)v wherein Ak is a C₁₋₂₀ alkylene chain in which one or more non-adjacent C atoms may be replaced with O, S, NR⁷, CO or COO; u is 0 or 1; Ar⁷ in each occurrence is independently an aromatic or heteroaromatic group which is unsubstituted or substituted with one or more substituents; and v is at least 1, optionally 1, 2 or 3.

Substituents of Ar⁷, if present, are preferably selected from F; Cl; NO₂; CN; and C₁₋₂₀ alkyl wherein one or more non-adjacent C atoms may be replaced with O, S, NR⁷, CO or COO and one or more H atoms may be replaced with F. Preferably, Ar⁷ is phenyl.

Preferably, R⁵⁵ is H or C₁₋₂₀ hydrocarbyl group.

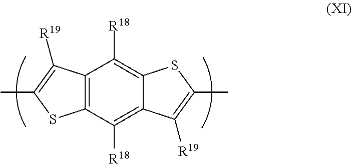

wherein R¹⁸ and R¹⁹ are each independently selected from H; F; C₁₋₁₂ alkyl wherein one or more non-adjacent, non-terminal C atoms may be replaced with O, S, COO or CO and one or more H atoms of the alkyl may be replaced with F; or an aromatic or heteroaromatic group, optionally phenyl or a 5-membered heteroaromatic group, which is unsubstituted or substituted with one or more substituents selected from F and C₁₋₁₂ alkyl wherein one or more non-adjacent, non-terminal C atoms may be replaced with O, S, COO or CO.

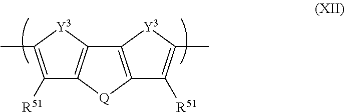

wherein Y³ is O, S or Se, preferably S; R⁵¹ is H or a substituent as described with reference to Formula (X), preferably H; and Q is C(R²¹) 2 or Si(R²¹) 2 wherein R²¹ in each occurrence is a substituent, preferably a substituent selected from: C₁₋₂₀ alkyl wherein one or more non-adjacent C atoms may be replaced with O, S, NR⁷, COO or CO and one or more H atoms of the alkyl may be replaced with F; and an aromatic or heteroaromatic group, preferably a C₆₋₂₀ aryl, more preferably phenyl, which is unsubstituted or substituted with one or more substituents. Substituents of an aromatic or heteroaromatic group R²¹ may be selected from R⁵¹ as described above.

Preferred acceptor units are selected from formulae (XIIIa), (XIIIb) and (XIIIc):

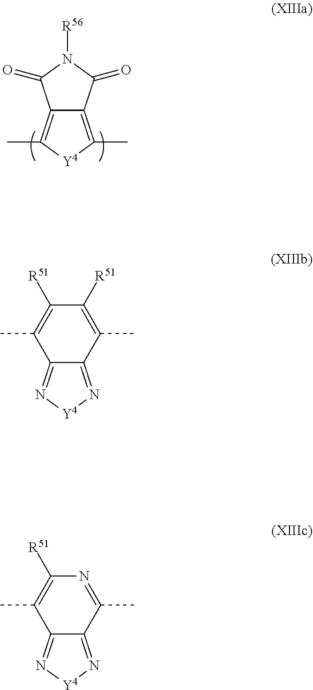

wherein R⁵⁶ is H or a substituent; R⁵¹ is H or a substituent as described above, preferably H or F; and Y⁴ is O, S or Se, preferably S.
R⁵⁶ is preferably H; C₁₋₁₂ alkyl wherein one or more C atoms of the C₁₋₁₂ alkyl other than a terminal C atom or the C atom bound to N of NR⁵⁶ may be replaced with O, S, CO or COO; or an aromatic or heteroaromatic group, preferably a C₆₋₁₂ aryl group, more preferably phenyl, which may be unsubstituted or substituted with one or more substituents.

Where present, substituents of an aromatic or heteroaromatic group R⁵⁶ are preferably selected from R⁵¹ as described above.

### Organic Electronic Device

A compound of formula (I) may be provided as an active layer of an organic electronic device. In a preferred embodiment, a photoactive layer of an organic photoresponsive device, more preferably an organic photodetector, comprises or consists of a compound of formula (I) as described herein and an electron-donating material.

In some embodiments, the photoactive layer is a bulk heterojunction layer comprising or consisting of a composition as described herein.

In some embodiments, the photoactive layer comprises two or more sub-layers including an electron-accepting sublayer comprising or consisting of a compound as described herein and an electron-donating sublayer comprising or consisting of an electron-donating material.

In some embodiments, the bulk heterojunction layer or electron-accepting sub-layer contains two or more electron-donating materials and/or two or more electron-accepting materials.

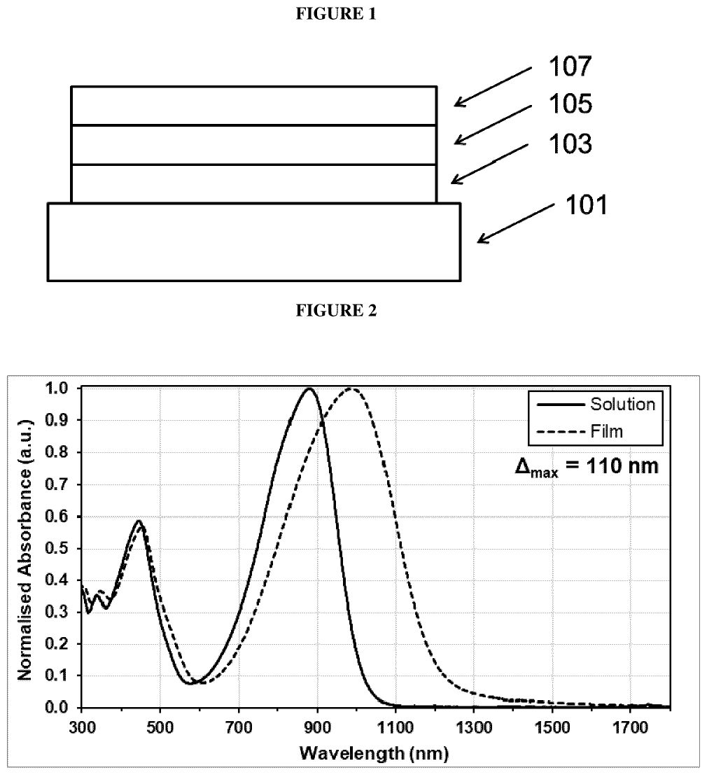

FIG. 1 illustrates an organic photoresponsive device according to some embodiments of the present disclosure. The organic photoresponsive device comprises a cathode 103, an anode 107 and a bulk heterojunction layer 105 disposed between the anode and the cathode. The organic photoresponsive device may be supported on a substrate 101, optionally a glass or plastic substrate.

Each of the anode and cathode may independently be a single conductive layer or may comprise a plurality of layers.

At least one of the anode and cathode is transparent so that light incident on the device may reach the bulk heterojunction layer. In some embodiments, both of the anode and cathode are transparent. The transmittance of a transparent electrode may be selected according to an emission wavelength of a light source for use with the organic photodetector.

FIG. 1 illustrates an arrangement in which the cathode is disposed between the substrate and the anode. In other embodiments, the anode may be disposed between the cathode and the substrate.

The organic photoresponsive device may comprise layers other than the anode, cathode and bulk heterojunction layer shown in FIG. 1. In some embodiments, a hole-transporting layer is disposed between the anode and the bulk heterojunction layer. In some embodiments, an electron-transporting layer is disposed between the cathode and the bulk heterojunction layer. In some embodiments, a work function modification layer is disposed between the bulk heterojunction layer and the anode, and/or between the bulk heterojunction layer and the cathode.

FIG. 1 illustrated herein comprises a bulk heterojunction photoactive layer 105. In other embodiments, photoactive layer 105 comprises two or more sub-layers including an electron-accepting sublayer comprising or consisting of a compound as described herein and an electron-donating sublayer comprising or consisting of an electron-donating material.

The area of the OPD may be less than about 3 cm², less than about 2 cm², less than about 1 cm², less than about 0.75 cm², less than about 0.5 cm² or less than about 0.25 cm². Optionally, each OPD may be part of an OPD array wherein each OPD is a pixel of the array having an area as described herein, optionally an area of less than 1 mm², optionally in the range of 0.5 micron²-900 micron².

The substrate may be, without limitation, a glass or plastic substrate. The substrate can be an inorganic semiconductor. In some embodiments, the substrate may be silicon. For example, the substrate can be a wafer of silicon. The substrate is transparent if, in use, incident light is to be transmitted through the substrate and the electrode supported by the substrate.

### Fullerene

In some embodiments, the compound of formula (I) is the only electron-accepting material of an electron-accepting sub-layer or a bulk heterojunction layer as described herein.

In some embodiments, an electron-accepting layer or a bulk heterojunction layer contains a compound of formula (I) and one or more further electron-accepting materials. Preferred further electron-accepting materials are fullerenes. The compound of formula (I): fullerene acceptor weight ratio may be in the range of about 1:0.1-1:1, preferably in the range of about 1:0.1-1:0.5.

Fullerenes may be selected from, without limitation, C₆₀, C₇₀, C₇₆, C₇₈ and C₈₄ fullerenes or a derivative thereof, including, without limitation, PCBM-type fullerene derivatives including phenyl-C₆₁-butyric acid methyl ester (C₆₀PCBM), TCBM-type fullerene derivatives (e.g., tolyl-C₆₁-butyric acid methyl ester (C₆₀TCBM)), and ThCBM-type fullerene derivatives (e.g., thienyl-C₆₁-butyric acid methyl ester (C₆₀ThCBM).

Fullerene derivatives may have formula (V):

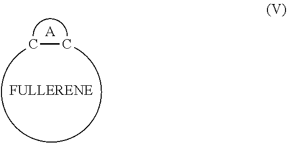

wherein A, together with the C-C group of the fullerene, forms a monocyclic or fused ring group which may be unsubstituted or substituted with one or more substituents.

Exemplary fullerene derivatives include formulae (Va), (Vb) and (Vc):

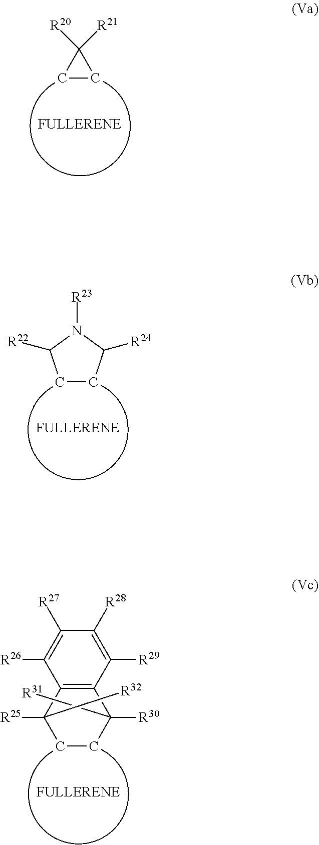

wherein R²⁰-R³² are each independently H or a substituent.

Substituents R²⁰-R³² are optionally and independently in each occurrence selected from the group consisting of aryl or heteroaryl, optionally phenyl, which may be unsubstituted or substituted with one or more substituents; and C₁₋₂₀ alkyl wherein one or more non-adjacent C atoms may be replaced with O, S, NR⁷, CO or COO and one or more H atoms may be replaced with F.

Substituents of aryl or heteroaryl, where present, are optionally selected from C₁₋₁₂ alkyl wherein one or more non-adjacent C atoms may be replaced with O, S, NR⁷, CO or COO and one or more H atoms may be replaced with F.

### Formulations

The photoactive layer of a photoresponsive device may be formed by any process including, without limitation, thermal evaporation and solution deposition methods.

Preferably, the photoactive layer comprising the compound of formula (I) is formed by depositing a formulation comprising or consisting of the electron-accepting material(s) and, in the case of a bulk heterojunction layer, the electron-donating material(s) dissolved or dispersed in a solvent or a mixture of two or more solvents. The formulation may be deposited by any coating or printing method including, without limitation, spin-coating, dip-coating, roll-coating, spray coating, doctor blade coating, wire bar coating, slit coating, ink jet printing, screen printing, gravure printing and flexographic printing.

The one or more solvents of the formulation may optionally comprise or consist of benzene or naphthalene substituted with one or more substituents selected from fluorine, chlorine, C₁₋₁₀ alkyl and C₁₋₁₀ alkoxy wherein two or more substituents may be linked to form a ring which may be unsubstituted or substituted with one or more C₁₋₆ alkyl groups, optionally toluene, xylenes, trimethylbenzenes, tetramethylbenzenes, anisole, indane and its alkyl-substituted derivatives, and tetralin and its alkyl-substituted derivatives.

The formulation may comprise a mixture of two or more solvents, preferably a mixture comprising at least one benzene substituted with one or more substituents as described above and one or more further solvents. The one or more further solvents may be selected from esters, optionally alkyl or aryl esters of alkyl or aryl carboxylic acids, optionally a C₁₋₁₀ alkyl benzoate, benzyl benzoate or dimethoxybenzene. In preferred embodiments, a mixture of trimethylbenzene and benzyl benzoate is used as the solvent. In other preferred embodiments, a mixture of trimethylbenzene and dimethoxybenzene is used as the solvent.

The formulation may comprise further components in addition to the electron-accepting material, the electron-donating material (in the case of a bulk heterojunction layer) and the one or more solvents. As examples of such components, adhesive agents, defoaming agents, deaerators, viscosity enhancers, diluents, auxiliaries, flow improvers colourants, dyes or pigments, sensitizers, stabilizers, nanoparticles, surface-active compounds, lubricating agents, wetting agents, dispersing agents and inhibitors may be mentioned.

### Applications

A circuit may comprise an organic photodetector as described herein connected to a voltage source for applying a reverse bias to the device and/or a device configured to measure photocurrent. The voltage applied to the photodetector may be variable. In some embodiments, the photodetector may be continuously biased when in use.

In some embodiments, a photodetector system comprises a plurality of photodetectors as described herein, such as an image sensor of a camera.

In some embodiments, a sensor may comprise an OPD as described herein and a light source wherein the OPD is configured to receive light emitted from the light source. In some embodiments, the light source has a peak wavelength of at least 900 nm or at least 1000 nm, optionally in the range of 900-1500 nm.

In some embodiments, the light from the light source may or may not be changed before reaching the OPD. For example, the light may be reflected, filtered, down-converted or up-converted before it reaches the OPD.

The organic photoresponsive device as described herein may be an organic photovoltaic device or an organic photodetector. An organic photodetector as described herein may be used in a wide range of applications including, without limitation, detecting the presence and/or brightness of ambient light and in a sensor comprising the organic photodetector and a light source. The photodetector may be configured such that light emitted from the light source is incident on the photodetector and changes in wavelength and/or brightness of the light may be detected, e.g., due to absorption by, reflection by and/or emission of light from an object, e.g., a target material in a sample disposed in a light path between the light source and the organic photodetector. The sample may be a non-biological sample, e.g., a water sample, or a biological sample taken from a human or animal subject. The sensor may be, without limitation, a gas sensor, a biosensor, an X-ray imaging device, an image sensor such as a camera image sensor, a motion sensor (for example for use in security applications) a proximity sensor or a fingerprint sensor. A 1D or 2D photosensor array may comprise a plurality of photodetectors as described herein in an image sensor. The photodetector may be configured to detect light emitted from a target analyte which emits light upon irradiation by the light source or which is bound to a luminescent tag which emits light upon irradiation by the light source. The photodetector may be configured to detect a wavelength of light emitted by the target analyte or a luminescent tag bound thereto.

### EXAMPLES

### Measurements

Unless stated otherwise, absorption spectra were measured using a Cary 5000 UV-VIS-NIR Spectrometer. Measurements were taken from 175 nm to 3300 nm using a PbSmart NIR detector for extended photometric range with variable slit widths (down to 0.01 nm) for optimum control over data resolution.

Unless stated otherwise, solution absorption data as provided herein is as measured in a methylated benzene solution, optionally a 1,2,4-trimethylbenzene solution.

Unless stated otherwise, HOMO and LUMO levels of materials as described herein are as measured by square wave voltammetry (SWV).

In SWV, the current at a working electrode is measured while the potential between the working electrode and a reference electrode is swept linearly in time. The difference current between a forward and reverse pulse is plotted as a function of potential to yield a voltammogram. Measurement may be with a CHI 660D Potentiostat.

The apparatus to measure HOMO or LUMO energy levels by SWV may comprise a cell containing 0.1 M tertiary butyl ammonium hexafluorophosphate in acetonitrile; a 3 mm diameter glassy carbon working electrode; a platinum counter electrode and a leak free Ag/AgCl reference electrode.

Ferrocene is added directly to the existing cell at the end of the experiment for calculation purposes where the potentials are determined for the oxidation and reduction of ferrocene versus Ag/AgCl using cyclic voltammetry (CV).

The sample is dissolved in toluene (3 mg/ml) and spun at 3000 rpm directly on to the glassy carbon working electrode.

LUMO=4.8−E ferrocene(peak to peak average)−E reduction of sample(peak maximum).

HOMO=4.8−E ferrocene(peak to peak average)+E oxidation of sample(peak maximum).

A typical SWV experiment runs at 15 Hz frequency; 25 mV amplitude and 0.004 V increment steps. Results are calculated from 3 freshly spun film samples for both the HOMO and LUMO data.

### Compound Example 1

Compound Example 1 was prepared according to the following scheme:

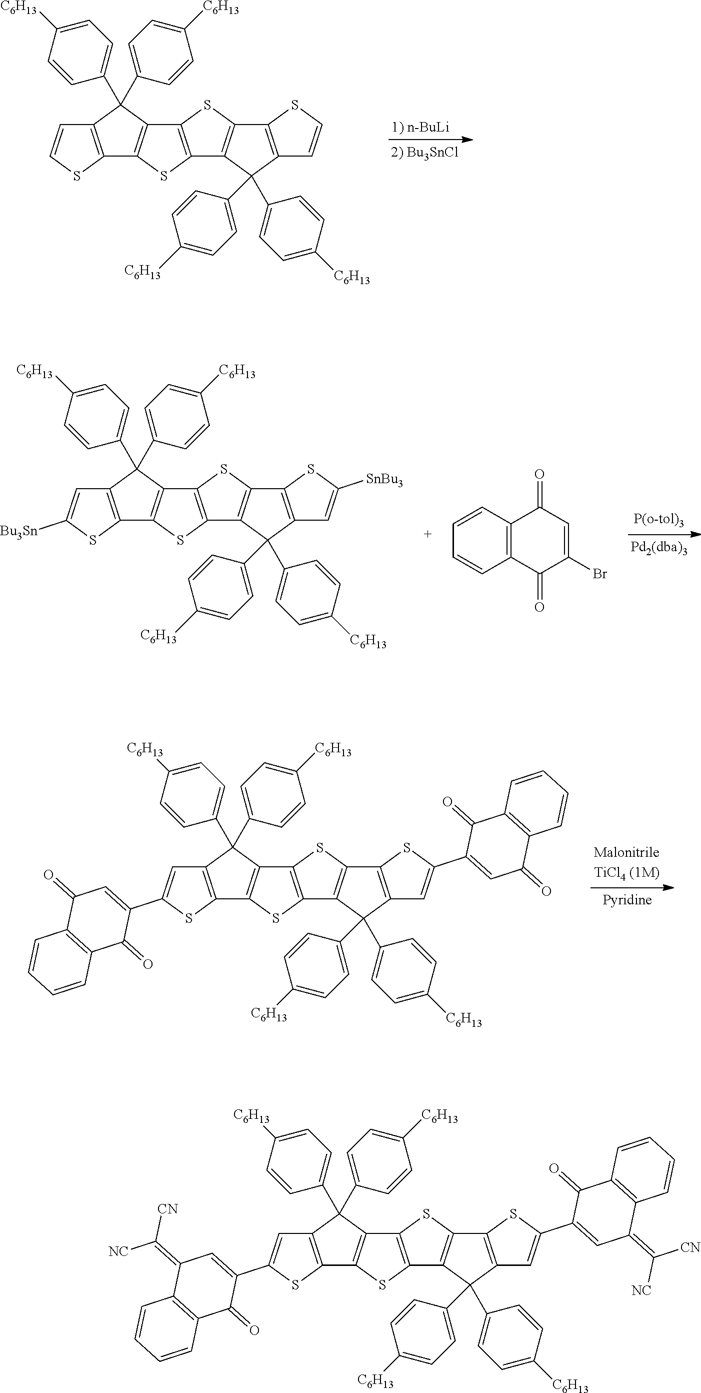

### Compound Example 1

FIG. 2 shows absorption spectra of a solution of Compound Example 1 in 1,2,4-trimethylbenzene and of a film of Compound Example 1 cast from 1,2,4-trimethylbenzene solution.

### Device Example 1

An organic photodetector having the following structure was prepared:

### Cathode/Donor: Acceptor Layer/Anode

A glass substrate coated with a 45 nm thick layer of indium-tin oxide (ITO) was treated with polyethyleneimine (PEIE) to modify the work function of the ITO.

A mixture of Donor Polymer 1 (donor): Compound Example 1 (non-fullerene acceptor): C₆₀PCBM (fullerene acceptor) in a mass ratio of 1:0.7:0.3 was deposited over the modified ITO layer by blade coating from a 10 mg/ml solution in 1,2,4 trimethylbenzene; 1,2-dimethoxybenzene 95:5 v/v solvent mixture. The film was dried at 80° C. to form a ca. 350 nm thick bulk heterojunction layer.

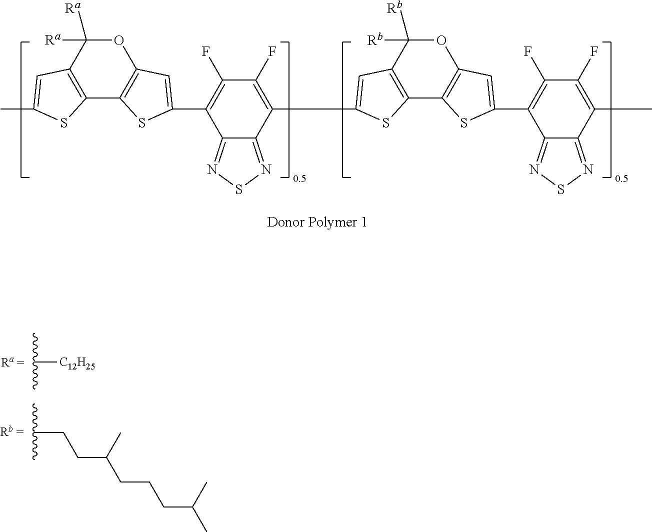

An anode stack of MoO₃ (10 nm) and Ag (100 nm) was formed over the bulk heterojunction by thermal evaporation.

### Device Example 2

Device Example 2 was prepared as described for Device Example 1 except that Donor Polymer 2 was used in place of Donor Polymer 1.

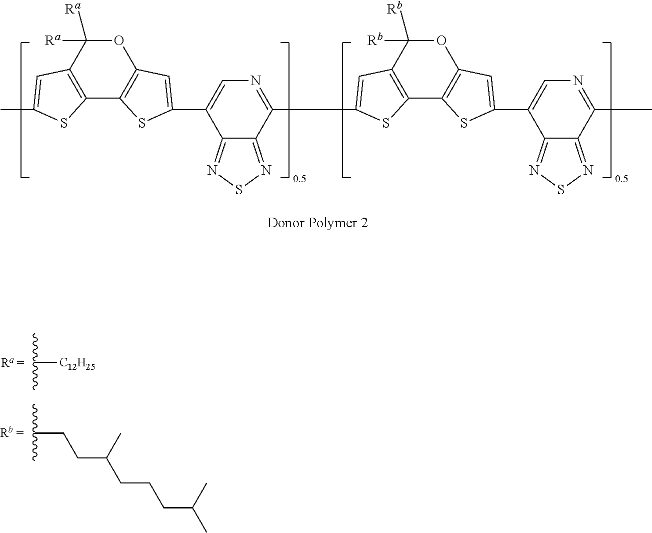

### Device Example 3

Device Example 3 was prepared as described for Device Example 1 except that Donor Polymer 3 was used in place of Donor Polymer 1.

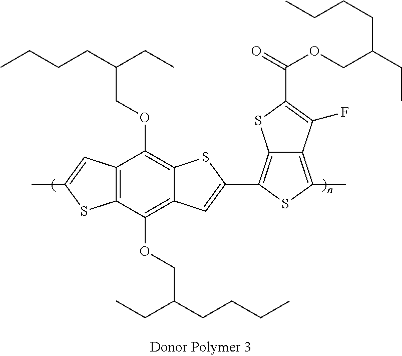 [SMILES: CCCCC(CC)COC(=O)c1sc2c(-c3cc4c(OCC(CC)CCCC)c5sc(C)cc5c(OCC(CC)CCCC)c4s3)sc(C)c2c1F]

External quantum efficiencies of Device Examples 1-3 were measured at −3V and are shown in FIG. 2.

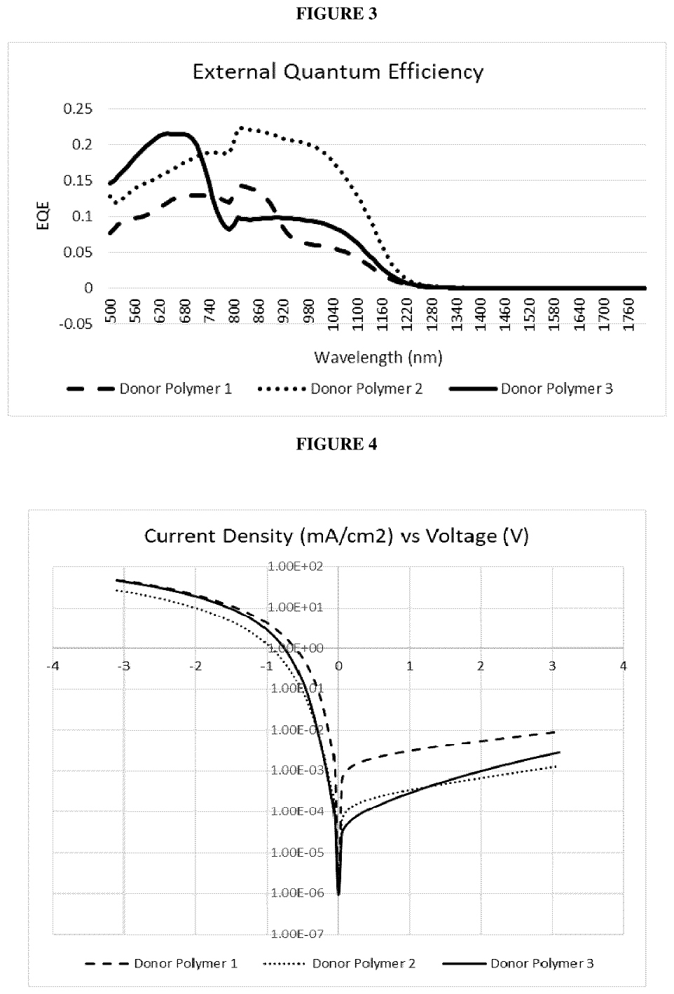

Diode characteristics (current density vs. voltage) of Device Examples 1-3 were measured for applied voltages from −3V to +3V and are shown in FIG. 3.

Specific detectivities vs wavelength of Device Examples 1-3 were measured and are shown in FIG. 4.

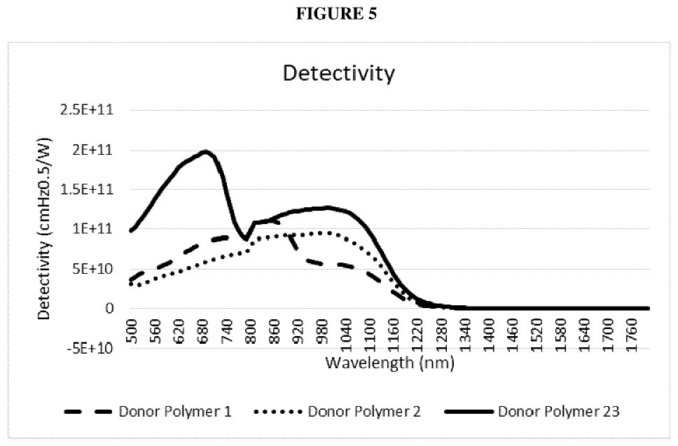

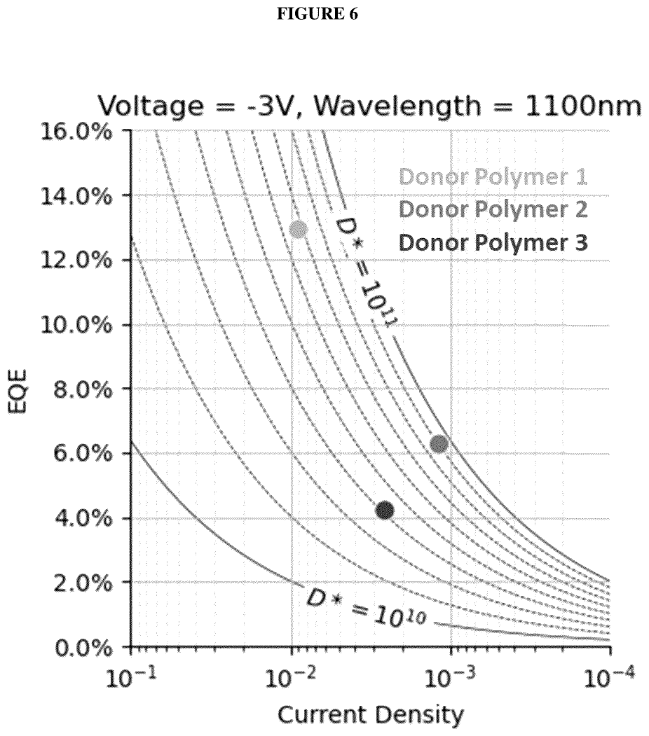

Due to the low dark current achieved at 1100 nm, a specific detectivity of greater than 1011 Jones was achieved, as shown in FIG. 5.

1. A compound of formula (I): 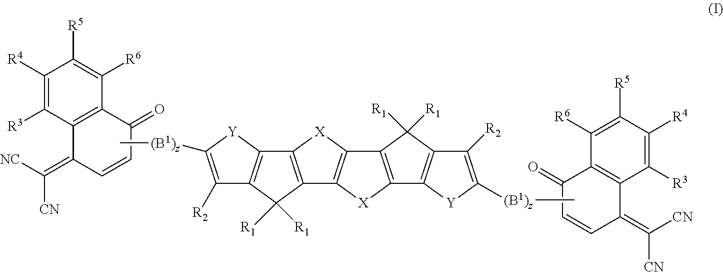
wherein:
R¹ in each occurrence is independently a substituent;
R² in each occurrence is H or substituent;
R³-R⁶ are each independently in each occurrence H or a halogen;
X in each occurrence is independently selected from is O, S and NR³ wherein R³ is H or a substituent;
Y in each occurrence is independently O or S;
B¹ independently in each occurrence is a bridging group; and
z in each occurrence is independently 0, 1, 2 or 3.

2. The compound according to claim 1 wherein R³ and R⁶ are each H.

3. The compound according to claim 1 wherein at least one of R⁴ and R⁵ is a halogen.

4. The compound according to claim 1 wherein R³-R⁶ are each independently in each occurrence selected from H, F and Cl.

5. The compound according to claim 1 wherein at least one z is at least 1.

6. The compound according to claim 5 wherein B¹ in each occurrence is selected from vinylene; thiophene; furan; thienothiophene; furofuran; and thienofuran.

7. The compound according to claim 1 wherein the compound of formula (I) is a compound of formula (Ia):

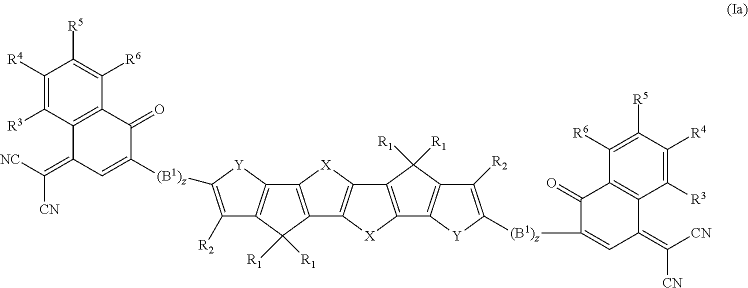

8. A composition comprising an electron-donating material and an electron-accepting material wherein the electron accepting material is a compound according to claim 1.

9. An organic electronic device comprising an active layer comprising a compound according to claim 1.

10. An organic electronic device comprising an active layer comprising a composition according to claim 9.

11. An organic electronic device according to claim 10 wherein the organic electronic device is an organic photoresponsive device and wherein the active layer is a photoactive disposed between the anode and cathode.

12. An organic electronic device according to claim 10 wherein the organic electronic device is an organic photoresponsive device and wherein the active layer is a photoactive disposed between the anode and cathode, wherein the photoactive layer is a bulk heterojunction layer comprising a composition according to claim 10.

13. An organic electronic device according to claim 12 wherein the organic photoresponsive device is an organic photodetector.

14. A photosensor comprising a light source and an organic photodetector according to claim 13 wherein the photosensor is configured to detect light emitted from the light source.

15. The photosensor according to claim 14, wherein the light source emits light having a peak wavelength of greater than 900 nm.

16. A formulation comprising a compound according to claim 1 dissolved or dispersed in one or more solvents.

17. A formulation comprising a compound according to claim 1 dissolved or dispersed in one or more solvents, wherein the compound according to claim 1 is an electron-accepting material and wherein the formulation further comprises an electron-donating material.

18. A method of forming an organic electronic device comprising an active layer comprising a compound according to claim 1, the method comprising deposition of a formulation comprising a compound according to claim 1 dissolved or dispersed in one or more solvents onto a surface and evaporation of the one or more solvents.
